(19) United States
(12) Patent Application Publication
(10) Pub. No.: US 2018/0369344 A1
(43) Pub. Date: Dec. 27, 2018

(54) Compositions and Methods for Treating Metabolic Disorders

(71) Applicant: YALE UNIVERSITY, New Haven, CT (US)

(72) Inventor: Arya MANI, Woodbridge, CT (US)

(21) Appl. No.: 15/781,606

(22) PCT Filed: Dec. 6, 2016
(86) PCT No.: PCT/US16/65150

§ 371 Date: Jun. 5, 2018

Related U.S. Application Data
(60) Provisional application No. 62/263,810, filed on Dec. 7, 2015.

(51) Int. Cl. A61K 38/48; A61P 9/10; A61P 3/10

(52) U.S. Cl. CPC A61K 38/482; A61K 9/0019; A61P 3/10; A61P 9/10

(57) ABSTRACT

The present invention relates to the unexpected discovery of an exceptionally non-conservative mutation in a very large kindred with a high prevalence of early CAD, obesity, hypertriglyceridemia and diabetes. The gene harboring the mutation is Cela2a. Characterization of the encoded protein unraveled important functions in regulation of insulin secretion and lipolysis. Thus, in various embodiments described herein, the invention encompasses a therapeutic composition comprising a Cela2a polypeptide or polynucleotide. Additionally, the invention relates to methods of altering glucose and/or lipid metabolism in a subject, methods of treating atherosclerosis, diabetes, hypertension, or hypertriglyceridemia in a subject, methods of detecting atherosclerosis and/or diabetes in a subject, and methods of identifying a subject at risk of developing atherosclerosis and/or diabetes. Further, the invention encompasses a kit for carrying out the aforementioned methods.

### CROSS REFERENCE TO RELATED APPLICATIONS

The present application claims priority under 35 U.S.C. § 119(e) to U.S. Provisional Patent Application No. 62/263,810, filed Dec. 7, 2015, which is incorporated herein by reference in its entirety.

### STATEMENT REGARDING FEDERALLY SPONSORED RESEARCH FOR DEVELOPMENT

This invention was made with government support under HL122822 and HL122830 awarded by National Institutes of Health. The government has certain rights in the invention.

### BACKGROUND OF THE INVENTION

Coronary artery disease (CAD) is the number one cause of mortality and morbidity and a major public health problem of our time. Epidemiologic studies have established the key roles of a cluster of metabolic risk factors in the pathogenesis of CAD and considerable work has been done to define the underlying molecular basis of individual risk factors. The most significant advances on these fronts in recent years have been the application of genetic studies for the identification of genes and pathways that contribute to rare form of these traits, mainly hyperlipidemia, diabetes and hypertension. This body of work has identified new targets for therapeutic intervention that are currently under development in the pharmaceutical industry. Despite these major efforts, the underlying pathogenesis of the most common form of metabolic risk factors in the general population and factors that underlie their clustering in the metabolic syndrome are not understood.

Tools that have emerged in the last decade culminating in whole exome/whole genome sequencing now permit identification of functional mutations underlying human disease without prior assumptions about disease pathogenesis or the pathways involved. A major benefit of this approach is that true causal relationships can be established between variations in genes and their cognate pathways and specific diseases of interest. These, in turn, provide fundamental new insight into disease pathogenesis, new diagnostic tools for identification of individuals with inherited susceptibility prior to onset of disease signs and symptoms, as well as logical starting points for identifying new targets and pathways for therapeutic intervention.

A successful experience in this path is the greatest testimony to this assertion. Identification of novel mutation in the Wnt coreceptor LRP6 as the first monogenic cause of CAD by our group (Mani et al, Science 2007, 315(5816):1278-82) changed the paradigm for the pathogenesis of CAD, type II diabetes (DM2) and metabolic syndrome. This finding led to unraveling many unknown functions of this gene and discovery of novel disease pathways. Exemplary findings include the function of LRP6 in ApoB binding and regulation of LDL/LDLR endocytosis (Liu, Mani, et al., Circ Res 2008, 103:1280-8; Ye, Mani, et al., J Biol Chem 2012;287:1335-44), regulation of growth factors and plasticity of vascular smooth muscle cells (Keramati, Mani, et al., Proc Natl Acad Sci U S A 2011, 108:1914-83), transcriptional regulation of the insulin receptor and regulation of nutrient sensing pathways and insulin signaling in the skeletal muscle (Singh, Mani, et al., Cell Metab 2013, 17:197-209), regulation of body fat and hepatic gluconeogenesis (Liu, Mani, et al., J Biol Chem 2012, 287:7213-23), hepatic IGF1/Akt/mTOR signaling, de novo hepatic lipogenesis, cholesterol synthesis and ApoB secretion (Go, Mani, et al., Cell Metab 2014, 19:209-20), fatty liver disease (Wang, Mani, et al., FASEB J 2015, 29(8):3436-45) and finally arterial wall protection (Srivastava, Mani, et al., Cell Rep 2015, 13:746-59). These findings have been all reproduced by lead scientists across the world and have laid the foundations for drug development targeting metabolic syndrome, CAD, and type II diabetes. Clearly, there is a need for new methods and compositions for therapeutic intervention in metabolic disorders, particularly metabolic disorders implicated in the development or progression of coronary artery disease (CAD) and type II diabetes. The current invention fulfills this need.

### SUMMARY OF THE INVENTION

In one aspect, the invention comprises a therapeutic composition comprising a Cela2a polypeptide in a pharmaceutically acceptable carrier. In one embodiment, the composition is formulated for intravenous (IV) administration.

In one aspect, the invention comprises an expression vector comprising a Cela2a polynucleotide.

In one embodiment the Cela2a polynucleotide is operably linked to a constitutive promoter or an inducible promoter.

In one embodiment, the invention comprises a host cell comprising the expression vector comprising a Cela2a polynucleotide, which may be operably linked to a constitutive promoter or an inducible promoter.

In one aspect, the invention comprises a therapeutic composition comprising a Cela2a polypeptide or polynucleotide in an intracellular delivery vehicle.

In one aspect, the invention comprises a method of altering glucose or lipid metabolism in a subject, the method comprising administering to the subject an effective amount of a composition comprising a Cela2a polypeptide or polynucleotide, thereby altering glucose or lipid metabolism in the subject.

In one embodiment, altering glucose or lipid metabolism comprises decreasing blood glucose level, stimulating insulin production, increasing adipogenesis, or reducing lipolysis.

In one aspect, the invention comprises a method of treating atherosclerosis, diabetes, hypertension, or hypertriglyceridemia in a subject, the method comprising administering to the subject an effective amount of a composition comprising a Cela2a polypeptide or polynucleotide, thereby treating atherosclerosis, diabetes, hypertension, or hypertriglyceridemia in the subject.

In one aspect, the invention comprises a kit comprising a capture reagent that binds a Cela2a polypeptide or polynucleotide.

In one embodiment, the kit further comprises a therapeutic composition comprising a Cela2a polypeptide in a pharmaceutically acceptable carrier.

In one aspect, the invention comprises a method of detecting atherosclerosis and/or diabetes in a subject, the method comprising measuring a level or a sequence of a Cela2a polypeptide or polynucleotide in a biological sample from the subject relative to a reference sequence, wherein a decreased level of the Cela2a polypeptide or polynucleotide or presence of a mutation in the Cela2a polypeptide or polynucleotide sequence indicates presence of atherosclerosis and/or diabetes in the subject.

In one aspect, the invention comprises a method of identifying a subject at risk of developing atherosclerosis and/or diabetes, the method comprising measuring a level or a sequence of a Cela2a polypeptide or polynucleotide in a biological sample from the subject relative to a reference level or reference sequence, wherein a decreased level of the Cela2a polypeptide or polynucleotide or presence of a mutation in the Cela2a polypeptide or polynucleotide sequence indicates the subject is at risk of developing atherosclerosis and/or diabetes.

In one aspect, the invention comprises a method of treating atherosclerosis and/or diabetes in a subject, the method comprising administering to the subject an effective amount of a Cela2a polypeptide, wherein the subject is pre-selected as having a mutation in a Cela2a polypeptide or polynucleotide relative to a reference sequence or a decreased level of a Cela2a polypeptide relative to a reference level, thereby treating atherosclerosis and/or diabetes in the subject.

In some embodiments, the sequence or mutation in the Cela2a polypeptide or polynucleotide is detected using a kit comprising a capture reagent that binds a Cela2a polypeptide or polynucleotide.

In some embodiments the step of measuring a sequence comprises measuring a sequence of a Cela2a polynucleotide.

In some embodiments the mutation is non-conservative.

In some embodiments the mutation is a loss-of-function mutation.

In some embodiments the biological sample is blood or plasma.

In some embodiments the composition is a therapeutic composition comprising a Cela2a polypeptide and a pharmaceutically acceptable carrier which may be formulated for intravenous (IV) administration and/or may include an intravenous delivery vehicle.

In some embodiments, the composition is administered to the subject by intravenous (IV) administration.

In some embodiments, the atherosclerosis is coronary artery disease (CAD) and/or the diabetes is type II diabetes.

In some embodiments, the subject is human.

In one aspect, the invention comprises a method of producing a Cela2a polypeptide, the method comprising (a) expressing a recombinant Cela2a polypeptide in a host cell, and (b) isolating the recombinant Cela2a polypeptide.

In some embodiments the host cell comprises an expression vector comprising a Cela2a polynucleotide, which may be operably linked to a constitutive promoter or an inducible promoter.

### BRIEF DESCRIPTION OF THE DRAWINGS

FIG. 1 is a diagram showing the pedigree structure of a kindred described herein. Dark gray symbols denote coronary artery disease (CAD) status. Symbols with triangles denotes subjects with metabolic syndrome who are too young to develop CAD. Light gray symbols denote unaffected subjects.

FIG. 2 is a plot showing glucose levels after intravenous (IV) administration of recombinant Cela2a protein in mice.

FIG. 3 is a plot depicting C-peptide levels before and 8 hours after IV administration of recombinant Cela2a protein in mice.

FIG. 4 is a plot showing insulin secretion in INS-1 cells in 2.5 and 9% glucose at 10 minutes (“10 min”) and 45 minutes (“45 min”) post protein stimulation. Adding mouse serum marginally reduced insulin secretion (“45 min+serum”). For each of the points “10 min,” “45 min,” and “45 min+serum,” the shaded bars from left to right indicate insulin secretion in cells in 2.5% glucose treated with a control, cells in 2.5% glucose treated with Cela2a protein, cells in 9% glucose treated with a control, and cells in 9% glucose treated with Cela2a protein, respectively.

FIGS. 5A-5B are images showing Cela2a and in vitro adipogenic differentiation. FIG. 5A is a micrograph showing oil red O staining demonstrating higher adipogenic transformation in 3T3L1 cells treated with recombinant Cela2a protein. FIG. 5B is a Western blot showing reduced levels of adipose triglyceride lipase (ATGL) and hormone-sensitive lipase (HSL) in Cela2a treated 3T3L1 cells.

FIG. 6 is a plot depicting triglyceride (TG) levels in lysates of 3T3L1 cells treated with recombinant Cela2a protein.

FIG. 7 is a plot showing glycerol levels in supernatant of 3T3L1 cells treated with recombinant Cela2a protein.

FIG. 8 is a plot showing that calcium signaling was higher in Cela2a treated cells compared with the related control samples.

FIGS. 9A-9C are plots showing that Cela2a inhibits ADP-induced platelet aggregation.

### DETAILED DESCRIPTION OF THE INVENTION

The present invention relates to the unexpected discovery of an exceptionally non-conservative mutation in a very large kindred with a high prevalence of early CAD, obesity, hypertriglyceridemia and diabetes. The gene harboring the mutation is Cela2a. Characterization of the encoded protein unraveled important functions in regulation of insulin secretion and lipolysis. Thus, in various embodiments described herein, the invention encompasses a therapeutic composition comprising a Cela2a polypeptide or polynucleotide. Additionally, the invention relates to methods of altering glucose and/or lipid metabolism in a subject, methods of treating atherosclerosis, diabetes, hypertension, or hypertriglyceridemia in a subject, methods of detecting atherosclerosis and/or diabetes in a subject, and methods of identifying a subject at risk of developing atherosclerosis and/or diabetes. Further, the invention encompasses a kit for carrying out the aforementioned methods.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. ”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, plasma, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes. In some embodiments, the biological sample is blood or plasma.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “Cela2a polypeptide” or “Cela2a protein” is meant a polypeptide or fragment thereof having at least 85% amino acid sequence identity to NCBI Accession No. NP_254275.1 and having elastase activity. The Cela2a polypeptide sequence provided at NCBI Accession No. NP_254275.1 is provided below:

SEQ ID NO: 1

| | |
|---|---|
| 1 | mirtlllstl vagalscgdp typpyvtrvv ggeearpnsw |
| | pwqvslqyss ngkwyhtcgg |
| 61 | slianswvlt aahcisssrt yrvglgrhnl yvaesgslav |
| | syskivvhkd wnsnqiskgn |
| 121 | diallklanp vsltdkiqla clppagtilp nnypcyvtgw |
| | grlqtngavp dvlqqgrllv |
| 181 | vdyatcsssa wwgssvktsm icaggdgvis scngdsggpl |
| | ncqasdgrwq vhgivsfgsr |
| 241 | lgcnyyhkps vftrvsnyid winsviann |

By “Cela2a polynucleotide” is meant a nucleic acid molecule encoding a Cela2a polypeptide. An exemplary Cela2a polynucleotide sequence is provided at NCBI Accession No. NM_033440.2, which is reproduced below:

SEQ ID NO: 2

| | |
|---|---|
| 1 | gcttacagaa ctcccacgga cacaccatga taaggacgct |
| | gctgctgtcc actttggtgg |
| 61 | ctggagccct cagttgtggg gaccccactt acccacctta |
| | tgtgactagg gtggttggcg |
| 121 | gtgaagaagc gaggcccaac agctggccct ggcaggtctc |
| | cctgcagtac agctccaatg |
| 181 | gcaagtggta ccacacctgc ggagggtccc tgatagccaa |
| | cagctgggtc ctgacggctg |
| 241 | cccactgcat cagctcctcc aggacctacc gcgtggggct |
| | gggccggcac aacctctacg |
| 301 | ttgcggagtc cggctcgctg gcagtcagtg tctctaagat |
| | tgtggtgcac aaggactgga |
| 361 | actccaacca aatctccaaa gggaacgaca ttgccctgct |
| | caaactggct aaccccgtct |
| 421 | ccctcaccga caagatccag ctggcctgcc tccctcctgc |
| | cggcaccatt ctacccaaca |
| 481 | actacccctg ctacgtcacg ggctggggaa ggctgcagac |
| | caacggggct gttcctgatg |
| 541 | tcctgcagca gggccggttg ctggttgtgg actatgccac |
| | ctgctccagc tctgcctggt |
| 601 | ggggcagcag cgtgaaaacc agtatgatct gtgctggggg |
| | tgatggcgtg atctccagct |
| 661 | gcaacggaga ctctggcggg ccactgaact gtcaggcgtc |
| | tgacggccgg tggcaggtgc |
| 721 | acggcatcgt cagcttcggg tctcgcctcg gctgcaacta |
| | ctaccacaag ccctccgtct |
| 781 | tcacgcgggt ctccaattac atcgactgga tcaattcggt |
| | gattgcaaat aactaaccaa |
| 841 | aagaagtccc tgggactgtt tcagacttgg aaaggtcaca |
| | gaaggaaaat aatataataa |
| 901 | agtgacaact atgcaaatca aaaaaaaaaa aaaa |

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include coronary artery disease (CAD), atherosclerosis, and type II diabetes.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence. The terms “polynucleotide” and “nucleic acid molecule” are used interchangeably herein.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis. The terms “polypeptide” and “protein” are used interchangeable herein.

By “marker” is meant any protein or polynucleotide having an alteration in expression level or activity that is associated with a disease or disorder. In some embodiments, decreased expression level or decreased activity (particularly, elastase activity) of Cela2a polypeptide is a marker for atherosclerosis, coronary artery disease, or type II diabetes.

By “metabolic syndrome” is meant a cluster of conditions, including without limitation, increased blood pressure (“hypertension”), increased blood glucose levels (“hyperglycemia”), and increased blood lipid levels (“hyperlipidemia”) (e.g., increased blood triglyceride levels (“hypertriglyceridemia”); increased blood cholesterol levels (“hypercholesterolemia”)), which occur together and increase risk of heart disease (e.g., coronary artery disease), atherosclerosis, and type II diabetes.

By “mutation” is meant a change in a polypeptide or polynucleotide sequence relative to a wild-type reference sequence. Exemplary mutations include point mutations, missense mutations, amino acid substitutions, and frameshift mutations. A mutation may be “conservative” or “non-conservative.” A “non-conservative” mutation is a mutation that results in alteration of an activity or function of the polypeptide. The alteration may be a decrease or increase in the activity or function of the polypeptide. A “loss-of-function mutation” is a mutation that decreases or abolishes an activity or function of a polypeptide. A “gain-of-function mutation” is a mutation that enhances or increases an activity or function of a polypeptide. In some embodiments, the mutation in Cela2a is a non-conservative mutation. In some other embodiments, the mutation in Cela2a is a loss-of-function mutation.

As used herein, “obtaining” as in “obtaining an agent” includes synthesizing, purchasing, or otherwise acquiring the agent.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one that expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/m1 ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Description

The present invention relates to discovery of an association between a non-conservative mutation in Cela2a and coronary artery disease (CAD), obesity, hypertriglyceridemia, and diabetes. Described herein are studies unraveling the novel functions of the disease gene Cela2a that underlies coronary artery disease, diabetes, and metabolic syndrome. Also described herein are its application in lowering blood glucose, plasma triglyceride, and atherosclerosis. The studies described herein focused on determining the underlying cause of a cluster of metabolic disorders that are associated with atherosclerosis and diabetes. The problem was approached at a genetic level. Results of the studies herein identified an exceptionally non-conservative mutation in a very large kindred with a high prevalence of early CAD, obesity, hypertriglyceridemia and diabetes. Characterization of the encoded protein unraveled important functions in regulation of insulin secretion and lipolysis.

The findings described herein indicate utility of Cela2a in treating coronary artery disease, diabetes, hypertension, hyperlipidemia. In particular, treatment of diabetes with Cela2a is expected to offer advantages over conventional diabetes treatment. Conventional diabetes drugs do not reduce cardiovascular mortality. Therapeutic interventions using Cela2a is expected to reduce cardiovascular mortality from hypertension and atherosclersosis by normalizing blood glucose and triglyceride levels and protecting the arterial wall.

### Molecular Genetics of Metabolic Syndrome and Characterization of a Gene (Cela2a) for Coronary Artery Disease (CAD) and Metabolic Syndrome

Metabolic syndrome (MetS) is a cluster of inherited risk factors for coronary artery disease (CAD) and type 2 diabetes. The factor(s) underlying the association of the risk factors remain largely unknown. Although highly heritable, mapping of the susceptibility genes for MetS has been constrained by the lack of perfect co-segregation of a genetic marker with inheritance of the trait, genetic heterogeneity, incomplete penetrance of the risk-allele, and high phenocopy rate. Investigation of rare Mendelian causes of MetS in outlier kindreds in conjunction with use of system biology by our group has been a powerful tool to circumvent these limitations and led to identification of several disease genes and their cognate disease pathways. By using this approach, mutations in Cela2a were identified, which underlie a cluster of traits featuring early onset CAD, type 2 diabetes, hypertension and hypertriglyceridemia.

Exome analysis in a large kindred with early onset atherosclerosis, hypertension, diabetes, and hypertriglyceridemia was carried out. Captured data was sequenced on the Illumina genome analyzer, followed by Image analysis and base calling. The resulting sequences were mapped to reference genome hg 19 using the Maqprogram SAMtools. Sequence data was then processed using Maq software. SAMtools software was used to detect single nucleotide variants (SNVs). The SNVs were then filtered out against reference genome hg 19. Filters were applied against a published database. A perl-based computer script was used to annotate variants based on protein effect, novelty, conservation and tissue expression. The analysis showed a single mutation that was shared among all affected subjects and was absent in unaffected subjects, in a previously characterized and functionally critical amino acid of the protein. This amino acid plays a crucial role in this protein and its substitution has shown previously to cause loss of function. In addition, all subjects with type 2 diabetes, hypertension or hypertriglyceridemia, who were too young to develop CAD were carriers of this mutation. This mutation is absent in all public and Yale Exome databases.

The characterization of the encoded protein in vitro and in vivo has led to striking insight into mechanism by which its altered function is linked to diverse traits. The protein is ubiquitously expressed and is present in the plasma. Its activity is reduced in the plasma of mutation carriers. In vivo administration of the protein results in significant decline in serum glucose and in vitro results in increased insulin secretion in INS1 cell lines. The recombinant protein increases adipogenesis in 3T3L1 cells by reducing baseline lipolysis.

The mechanisms by which this protein protects against diabetes, hypertriglyceridemia and atherosclerosis in vivo are not known. Elucidating the function of this protein in vivo is a critical step in understanding the function of the protein and how it can be therapeutically utilized.

### Methods of Treatment

As described herein, Cela2a polypeptides are useful for treating atherosclerosis (in particular, coronary artery disease (CAD)) and/or type II diabetes in a subject. Accordingly, the present invention provides methods of treating atherosclerosis, coronary artery disease (CAD), diabetes, hypertension, and/or hyperlipidemia or symptoms thereof which comprise administering a therapeutically effective amount of a pharmaceutical composition comprising a Cela2a polypeptide or polynucleotide herein to a subject (e.g., a mammal such as a human). One embodiment is a method of treating a subject suffering from or susceptible to a metabolic syndrome, coronary artery disease (CAD), atherosclerosis, diabetes (e.g., type II diabetes), hyperglycemia, hypertension, hyperlipidemia (e.g., hypertriglyceridemia), or disorder or symptom thereof. The method includes the step of administering to the mammal a therapeutic amount of a Cela2a polypeptide or polynucleotide herein sufficient to treat atherosclerosis, coronary artery disease (CAD), diabetes, or a disorder or symptom thereof, under conditions such that the disease or disorder is treated. In some embodiments, the Cela2a polypeptide is a recombinant Cela2a polypeptide.

In another aspect, the invention comprises a method of altering glucose or lipid metabolism in a subject, the method comprising administering to a subject an effective amount of a composition comprising a Cela2a polypeptide or polynucleotide, thereby altering glucose or lipid metabolism in the subject. As demonstrated in the figures and examples, Cela2a polypeptide or polynucleotide has a range of physiological effects including decreasing blood glucose level, stimulating insulin production, increasing adipogenesis, and reducing lipolysis. Accordingly, in certain embodiments, altering glucose or lipid metabolism comprises decreasing blood glucose level, stimulating insulin production, increasing adipogenesis, and reducing lipolysis.

The methods herein include administering to the subject (including a subject identified as in need of such treatment) an effective amount of a Cela2a polypeptide or polynucleotide described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the polypeptides or polynucleotides herein, such as a Cela2a polypeptide or polynucleotide to a subject (e.g., animal, human) in need thereof, including a mammal, particularly a human. Such treatment will be suitably administered to subjects, particularly humans, suffering from, having, susceptible to, or at risk for atherosclerosis, coronary artery disease (CAD), diabetes, or a disease, disorder, or symptom thereof. Determination of those subjects “at risk” can be made by any objective or subjective determination by a diagnostic test or opinion of a subject or health care provider (e.g., genetic test, enzyme or protein marker (e.g., expression level or activity of Cela2a), family history, and the like). The Cela2a polypeptides or polynucleotides herein may be also used in the treatment of any other disorders in which metabolic syndrome, atherosclerosis, coronary artery disease (CAD), diabetes, hypertension, and/or hyperlipidemia may be implicated.

In one embodiment, the invention provides a method of monitoring treatment progress. The method includes the step of determining a level of diagnostic marker (Cela2a polypeptide or polynucleotide) or diagnostic measurement (e.g., screen, assay) in a subject suffering from or susceptible to a disorder or symptoms thereof associated with atherosclerosis, coronary artery disease (CAD), diabetes, hypertension, and/or hyperlipidemia, in which the subject has been administered a therapeutic amount of a compound herein sufficient to treat the disease or symptoms thereof. The level of Cela2a polypeptide or polynucleotide determined in the method can be compared to known levels of Cela2a polypeptide or polynucleotide in either healthy normal controls or in other afflicted patients to establish the subject's disease status. In preferred embodiments, a second level of Cela2a polypeptide or polynucleotide in the subject is determined at a time point later than the determination of the first level, and the two levels are compared to monitor the course of disease or the efficacy of the therapy. In certain preferred embodiments, a pre-treatment level of Cela2a polypeptide or polynucleotide in the subject is determined prior to beginning treatment according to this invention; this pre-treatment level of Cela2a polypeptide or polynucleotide can then be compared to the level of Cela2a polypeptide or polynucleotide in the subject after the treatment commences, to determine the efficacy of the treatment.

### Recombinant Cela2a Polypeptide Expression

The invention provides recombinant Cela2a polypeptides, which are useful for treating atherosclerosis (in particular, coronary artery disease (CAD)) and/or type II diabetes in a subject. When administered to a subject, the Cela2a polypeptides of the invention alter lipid and/or glucose metabolism in the subject, thereby treating or lowering the risk of atherosclerosis and/or diabetes in the subject. In some embodiments, administration of a Cela2a polypeptide to a subject decreased plasma glucose levels in the subject. In some other embodiments, administration of a Cela2a polypeptide to a subject stimulated insulin secretion. In still other embodiments, administration of a Cela2a polypeptide increased adipogenesis and/or decreased lipolysis.

Recombinant polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle. Accordingly, the invention provides methods of producing a polypeptide of the invention, the method comprising (a) heterologously expressing an expression vector comprising a polynucleotide encoding the polypeptide in a host cell; and (b) isolating the polypeptide from the host cell. The invention further provides expression vectors comprising a polynucleotide encoding a Cela2a polypeptide, as well as host cells comprising these expression vectors.

In one aspect, the invention comprises an expression vector comprising a Cela2a polynucleotide. In some embodiments, the Cela2a polynucleotide is operably linked to a constitutive or inducible promoter. In some embodiments, the invention comprises a host cell containing these expression vectors. In one aspect the invention comprises a method of producing a Cela2a polypeptide, the method comprising expressing a recombinant Cela2a polypeptide in a host cell and isolating the recombinant Cela2a polypeptide. Host cells, methods of expression and methods of isolating the recombinant polypeptide are discussed below.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

In some embodiments, the polypeptides of the invention are produced in a bacterial expression system. One particular bacterial expression system for polypeptide production is the E. coli pET expression system (e.g., pET-28) (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa. Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. In some embodiments, to facilitate purification of the recombinant Cela2a polypeptide, the polypeptide comprises an epitope tag fused to Cela2a polypeptide. The polypeptide is then isolated using an antibody against the epitope tag. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column. Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Methods of Delivery

Cela2a polypeptides or polynucleotides of the invention, which are useful for treating atherosclerosis (e.g., coronary artery disease (CAD), type II diabetes, hyperglycemia, or hypertriglyceridemia in an organism, may be delivered to an organism in any manner. Cela2a polypeptides of the invention, for example, may be administered intravenously, into an organism's bloodstream. Cela2a is known as a pancreatic elastase. The findings described herein showed variable expression of Cela2a in all tissues. Thus, in some embodiments, Cela2a may be further delivered intracellularly to particular cells or tissues of an organism. The Cela2a polypeptide may be delivered to cells as a polypeptide. Alternatively, a polynucleotide encoding an amino acid sequence of the Cela2a polypeptide may be delivered to cells for heterologous expression of a Cela2a polypeptide in the cells. Thus, the present invention features polypeptides delivered to a cell by contacting the cell with a composition comprising the polypeptide or by heterologously expressing the polypeptide in the cell.

### Intracellular Delivery of Polypeptides

In some embodiments, the Cela2a polypeptide of the invention is delivered intracellularly to cells of a subject. The Cela2a polypeptide is delivered to the cells of the subject in a form in which they can be taken up so that therapeutically effective levels of the polypeptide, or fragment thereof, is in functional form in the cells.

Methods of intracellular delivery of polypeptides are known to one of skill in the art. Exemplary methods of intracellular delivery of polypeptides include, without limitation, incorporation of the polypeptide into a liposome. Liposomes are phospholipid vesicles with sizes varying from 50 to 1000 nm, which can be loaded with polypeptides or other agents. Liposomal intracellular delivery of polypeptides into cells typically relies on endocytosis of the liposome-encapsulated polypeptide into the cell. Examples of suitable liposomes for intracellular delivery of polypeptides may be pH-sensitive liposomes. Such liposomes are made of pH-sensitive components; after being endocytosed in intact form, the liposome fuses with the endovacuolar membrane under lowered pH inside the endosome and destabilizes it, thereby releasing the contents (including the polypeptides encapsulated in the liposome) into the cytoplasm. The liposomes may also be further modified to enhance their stability or lifetime during circulation (e.g., by PEGylated liposomes). Liposomes may also be modified to specifically target antigens (e.g., “immunoliposomes” or liposomes embedded with antibodies to an antigen). Antibody-bearing liposomes may have the advantages of targetability and facilitated uptake via receptor-mediated endocytosis.

Other methods of intracellular delivery of polypeptides include, without limitation, use of cell penetrating peptides (CPPs). A cell penetrating peptide or “CPP” is a protein or peptide that can translocate through cellular membranes. A polypeptide for delivery into a cell is fused with a CPP, thereby enabling or enhancing delivery of the polypeptide fusion into the cell. Cell penetrating peptides include, for example, a trans-activating transcriptional activator (TAT) from HIV-1, Antenapedie (Antp, a transcription factor in Drosophila), and VP22 (a herpes virus protein).

Another exemplary method for intracellular delivery of polypeptides of the invention is the use of supercharged proteins. Supercharged proteins or supercharged polypeptides are a class of engineered or naturally existing polypeptides having an unusually high positive or negative net theoretical charge. Membranes of cells are typically negatively charged. Superpositively charged polypeptides are able to penetrate cells (particularly mammalian cells), and associating cargo with superpositively charged polypeptides (e.g., polypeptides or polynucleotides) can enable functional delivery of these macromolecules into cells, in vitro or in vivo. Methods of generating supercharged polypeptides and using supercharged polypeptides for intracellular polypeptide delivery are described in further detail in, for example, Zuris et al. Nat. Biotechnol. (2015) 33:73-80 and Liu et al. Methods Enzymol. 2012, 503: 293-319.

Accordingly, in some aspects, the invention provides a Cela2a polypeptide fused to a polypeptide enabling intracellular delivery of the Cela2a polypeptide (e.g., a cell penetrating peptide or supercharged polypeptide). In other aspects, the invention provides a composition comprising Cela2a polypeptides in a vehicle for intracellular delivery (e.g., a liposome).

### Polynucleotide Therapy

Another therapeutic approach for treating atherosclerosis (e.g., coronary artery disease), diabetes, hyperglycemia, or hypertriglyceridemia is polynucleotide therapy using a polynucleotide encoding a Cela2a polypeptide of the invention, or a biologically active fragment thereof. Thus, provided herein are isolated polynucleotides encoding a Cela2a polypeptide of the invention, or fragment thereof. Expression of such polynucleotides or nucleic acid molecules in a cell or organism is expected to treat atherosclerosis (e.g., coronary artery disease), diabetes, hyperglycemia, or hypertriglyceridemia in the subject. Such nucleic acid molecules can be delivered to cells of a subject having a metabolic disease or disorder such as atherosclerosis (e.g., coronary artery disease), diabetes, metabolic syndrome, hyperglycemia, hypertension, hyperlipidemia, or hypertriglyceridemia. The nucleic acid molecules must be delivered to the cells of a subject in a form in which they can be taken up so that therapeutically effective levels of the Cela2a polypeptide, or fragment thereof, can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a Cela2a polypeptide of the invention, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding a Cela2a polypeptide (or fragment thereof) systemically.

Non-viral approaches can also be employed for the introduction of the therapeutic to a cell of a patient requiring suppression of a neurological disease. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of genes encoding Cela2a polypeptides into the affected tissues of a patient can also be accomplished by transferring a nucleic acid encoding the Cela2a polypeptide into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Delivery of polynucleotides of the invention may also include or be performed in combination with gene or genome editing methods, such as CRISPR-Cas systems, to introduce polynucleotides encoding Cela2a polypeptides in cells. Gene or genome editing methods such as CRISPR-Cas systems are further described in for example, Sander et al. (2014), Nature Biotechnology 32, 347-355; Hsu et al. (2014), Cell 157(6): 1262-1278.

### Pharmaceutical Compositions

The present invention features compositions useful for treating metabolic diseases or disorders, such as atherosclerosis (e.g., coronary artery disease), diabetes (e.g. type II diabetes), hyperglycemia, hypertension, metabolic syndrome, or hyperlipidemia (e.g., hypertriglyceridemia) in a subject. In some embodiments, the composition comprises a Cela2a polypeptide or a biologically active fragment thereof and a pharmaceutically acceptable carrier. In some embodiments, the pharmaceutical composition is formulated for intravenous (IV) administration. In some other embodiments, the composition comprises a polynucleotide encoding an amino acid sequence of the Cela2a or a biologically active fragment thereof.

The administration of a composition comprising a Cela2a polypeptide or polynucleotide described herein for the treatment of a metabolic disease or disorder may be by any suitable means that results in a concentration of the therapeutic that, combined with other components, is effective in ameliorating, reducing, or stabilizing the disease or disorder in a subject. The composition may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the agent in the patient. The amount of the therapeutic agent to be administered varies depending upon the manner of administration, the age and body weight of the patient, and with the clinical symptoms of the cancer. Generally, amounts will be in the range of those used for other agents used in the treatment of metabolic disease or disorders such as atherosclerosis (e.g., coronary artery disease), diabetes (e.g. type II diabetes), hyperglycemia, hypertension, metabolic syndrome, or hyperlipidemia (e.g., hypertriglyceridemia) or other diseases associated with Cela2a, although in certain instances lower amounts will be needed because of the increased specificity of the agent. A composition is administered at a dosage that appropriately alters glucose and/or lipid metabolism (e.g., decreases plasma glucose levels) or that otherwise treats atherosclerosis (e.g., coronary artery disease), diabetes (e.g. type II diabetes), hyperglycemia, hypertension, metabolic syndrome, or hyperlipidemia (e.g., hypertriglyceridemia) as determined by a method known to one skilled in the art.

The Cela2a polypeptide or polynucleotide may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with a tumor; (v) formulations that allow for convenient dosing, such that doses are administered, for example, once every one or two weeks; and (vi) formulations that target a cancer using carriers or chemical derivatives to deliver the therapeutic agent to a particular cell type (e.g., cancer cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cancer, the composition may include suitable parenterally acceptable carriers and/or excipients. The active therapeutic agent(s) (e.g., an antibody mimic, monobody, or polynucleotide described herein) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic (i.e., a Cela2a polypeptide or polynucleotide herein) is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Diagnostics

The present invention further provides a number of diagnostic assays that are useful for early detection of a metabolic disease or disorder, such as atherosclerosis (e.g., coronary artery disease (CAD)), or diabetes (e.g. type II diabetes), in a subject. The studies described herein identified an exceptionally non-conservative mutation in Cela2a in a very large kindred with a high prevalence of early CAD, obesity, hypertriglyceridemia and diabetes. Accordingly, the present invention provides Cela2a as a marker for the detection of atherosclerosis and/or diabetes. In some aspects, the invention features a method of detecting atherosclerosis and/or diabetes in a subject. In some other aspects, the invention features a method of identifying a subject at risk of developing atherosclerosis and/or diabetes. The methods comprise measuring a level or a sequence of a Cela2a polypeptide or polynucleotide in a biological sample from the subject relative to a reference sequence, wherein a decreased level of the Cela2a polypeptide or polynucleotide or presence of a mutation in the Cela2a polypeptide or polynucleotide sequence indicates presence of atherosclerosis and/or diabetes in the subject.

In some embodiments, a subject is selected for treatment with a Cela2a polypeptide or polynucleotide of the invention by detection of a mutation in Cela2a in a biological sample obtained from the subject. The biological sample may be blood or plasma. Methods for detecting a Cela2a mutation in the sample include immunoassay, direct sequencing, and probe hybridization to a polynucleotide encoding the mutant polypeptide. In other embodiments, a subject is selected for treatment with a Cela2a polypeptide or polynucleotide of the invention by detection of a decreased level of Cela2a polypeptide or polynucleotide in a biological sample obtained from the subject. In various embodiments, any of these methods may be performed using the kits described below. In various embodiments, the methods comprise measuring a sequence for Cela2a polynucleotide. In some embodiments, the mutation may be a non-conservative mutation. In some embodiments the mutation may be a loss of function mutation. Methods for detecting a level of Cela2a polypeptide or polynucleotide in a subject are described further herein.

The presence or absence of the herein disclosed marker(s) (e.g., Cela2a polypeptide or polynucleotide) is measured in a biological sample from a subject. Biological samples that are used to evaluate the presence or absence of the herein disclosed markers include without limitation blood, serum, plasma, urine. In one embodiment, the biological sample is plasma or blood.

While the examples provided below describe specific methods of detecting levels of these markers, the skilled artisan appreciates that the invention is not limited to such methods. The biomarkers of this invention can be detected by any suitable method. For example, marker levels are quantifiable by any standard method, such methods include, but are not limited to real-time PCR, Southern blot, PCR, mass spectroscopy, and/or antibody binding.

The methods described herein can be used individually or in combination with other markers for a more accurate detection of the biomarkers (e.g., immunoassay, mass spectrometry, and the like). The accuracy of a diagnostic assay may characterized by a Receiver Operating Characteristic curve (“ROC curve”). An ROC is a plot of the true positive rate against the false positive rate for the different possible cutpoints of a diagnostic test. An ROC curve shows the relationship between sensitivity and specificity. That is, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test, while an area of 0.5 represents a less useful test. In certain embodiments, biomarkers and diagnostic methods of the present invention have an AUC greater than 0.50. In other embodiments, biomarkers and diagnostic methods of the present invention have an AUC greater than 0.60. In other embodiments, biomarkers and diagnostic methods of the present invention have an AUC greater than 0.70.

In particular embodiments, the biomarker of the invention (Cela2a polypeptide or polynucleotide) is measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, and chemiluminescence. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead based microarrays (suspension arrays).

In particular embodiments, the immunoassay is carried out using multiplexed bead assays. In particular embodiments, the immunoassay is carried out using magnetic bead-based multiplexed assays. Multiplexed bead assays use a series of spectrally discrete particles that are used to capture and quantitate soluble analytes. The analyte is then measured by detection of a fluorescence-based emission and flow cytometric analysis. Multiplexed bead assays generate data that is comparable to ELISA based assays, but in a multiplexed or simultaneous fashion. Concentration of unknowns is calculated for the cytometric bead array as with any sandwich format assay, i.e., through the use of known standards and by plotting unknowns against a standard curve. Further, multiplexed bead assays allow quantification of soluble analytes in samples never previously considered due to sample volume limitations. In addition to the quantitative data, powerful visual images are generated revealing unique profiles or signatures that provide the user with additional information at a glance.

In particular embodiments, subjects are characterized as having decreased level of Cela2a polypeptide or polynucleotide. In other embodiments, subjects are characterized as having a mutation in a Cela2a polypeptide or polynucleotide. In some embodiments, the mutation in the Cela2a polypeptide or polynucleotide is non-conservative. In still other embodiments, the mutation is a loss-of-function mutation.

In particular embodiments, the level of a marker (e.g., Cela2a polypeptide or polynucleotide) is compared to a reference. In one embodiment, the reference is the level of marker present in a control sample obtained from a patient that does not have a pancreatic cancer. In some examples of the disclosed methods, when the level of expression of a biomarker(s) is assessed, the level is compared with the level of expression of the biomarker(s) in a reference standard. By reference standard is meant the level of expression of a particular biomarker(s) from a sample or subject lacking a metabolic disease or disorder (e.g., atherosclerosis, coronary artery disease (CAD), type II diabetes, hypertension, metabolic syndrome, hyperglycemia, or hyperlipidemia (e.g., hypertriglyceridemia)) or in the absence of a particular variable such as a therapeutic agent. Alternatively, the reference standard comprises a known amount of biomarker. Such a known amount correlates with an average level of subjects lacking a cancer, at a selected stage of the metabolic disease or condition, or in the absence of a particular variable such as a therapeutic agent. A reference standard also includes the expression level of one or more biomarkers from one or more selected samples or subjects as described herein. For example, a reference standard includes an assessment of the expression level of one or more biomarkers in a sample from a subject that does not have a metabolic disease or disorder (e.g., atherosclerosis, coronary artery disease (CAD), type II diabetes, hypertension, metabolic syndrome, hyperglycemia, or hyperlipidemia (e.g., hypertriglyceridemia)), is at a selected stage of progression of the disease or disorder, or has not received treatment for the disease or disorder. Another exemplary reference standard includes an assessment of the expression level of one or more biomarkers in samples taken from multiple subjects that do not have a metabolic disease or disorder (e.g., atherosclerosis, coronary artery disease (CAD), type II diabetes, hypertension, metabolic syndrome, hyperglycemia, or hyperlipidemia (e.g., hypertriglyceridemia)), are at a selected stage of progression of the disease or disorder, or have not received treatment for the metabolic disease or disorder.

### Combination Therapies

Optionally, an anti-metabolic disease or anti-metabolic disorder therapeutic of the invention (e.g., a Cela2a polynucleotide or polypeptide as described herein) may be administered in combination with any other standard treatment or therapy for a metabolic disease or disorder (e.g., a metabolic disease or disorder such as atherosclerosis, coronary artery disease (CAD), type II diabetes, hypertension, metabolic syndrome, hyperglycemia, or hyperlipidemia (e.g., hypertriglyceridemia); such methods are known to the skilled artisan and described in Remington's Pharmaceutical Sciences by E. W. Martin.

### Kits

The invention provides kits for the treatment, prevention, or detection of a metabolic disease or disorder, particularly atherosclerosis, coronary artery disease (CAD), type II diabetes, hypertension, metabolic syndrome, hyperglycemia, or hyperlipidemia (e.g., hypertriglyceridemia). In one embodiment, the kit includes a therapeutic or prophylactic composition containing an effective amount of a Cela2a polypeptide, or fragment thereof (or a polynucleotide encoding such) in unit dosage form. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. In some other embodiments, the kit comprises reagents detecting a mutation associated with a metabolic disease or disorder such as atherosclerosis, coronary artery disease (CAD), type II diabetes, hypertension, metabolic syndrome, hyperglycemia, or hyperlipidemia (e.g., hypertriglyceridemia) in a subject. For example, the reagents may be primers or hybridization probes for detection of mutation in Cela2a or detection of expression levels of Cela2a. In still other embodiments, the kit comprises detection reagents for detection of the sequence or level of a Cela2a polypeptide or polynucleotide together with a therapeutic or prophylactic composition containing an effective amount of a Cela2a polypeptide or polynucleotide.

In one aspect the invention comprises a capture reagent that binds Cela2a polypeptide or polynucleotide. In some embodiments, the kit further comprises a therapeutic composition comprising a Cela2a polypeptide in a pharmaceutically acceptable carrier.

If desired a composition comprising a therapeutic agent of the invention (e.g., a Cela2a polypeptide or polynucleotide described herein) is provided together with instructions for administering the agent to a subject having or at risk of developing a neurological disease. The instructions will generally include information about the use of the composition for the treatment or prevention of neurological disease. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Identification of Common Alleles Through Discovery of a Rare Founder Mutation

In the case of monogenic diseases, the available genetic tools are extremely powerful. However, in the case of a typical complex trait like coronary artery disease (CAD), type II diabetes (DM2) and metabolic syndrome, the situation is vastly more complicated. The number of genes that influence these traits and the magnitude of their effects in single individuals cannot be estimated with any precision. Thus, the same pattern of familial recurrence of disease can be produced by the combined actions of as few as 2 or 3 genes. This uncertainty about the underlying model of inheritance influencing the trait imposes serious methodological problems for study design. In the setting of high degrees of genetic heterogeneity, investigation of the general population may prove highly problematic and costly, as the proper scale of a study is likely impossible to predict. In such a setting, the likelihood of success can be greatly improved by increasing the genetic homogeneity, such that there are more likely to be single genes with large effect.

The approach taken herein was to systematically study the families of individuals at the extreme ends of quantitative distributions by selection for increased disease severity and early age of disease onset. This approach presumes that by biasing for extreme phenotypes, the likelihood of identifying genes with major effects is increased. This approach is particularly powerful in identification of founder mutations in outlier kindreds, where a single or small number of disease alleles are among the gene pool of a small genetically isolated population that rapidly expands. Taking this approach, an exceptionally nonconservative mutation in a very large kindred with a high prevalence of early CAD, obesity, hypertriglyceridemia and diabetes was identified. Characterization of the encoded protein unraveled important functions in regulation of insulin secretion and lipolysis. Independent mutations in this gene were subsequently found in unrelated cases.

### Preliminary Results

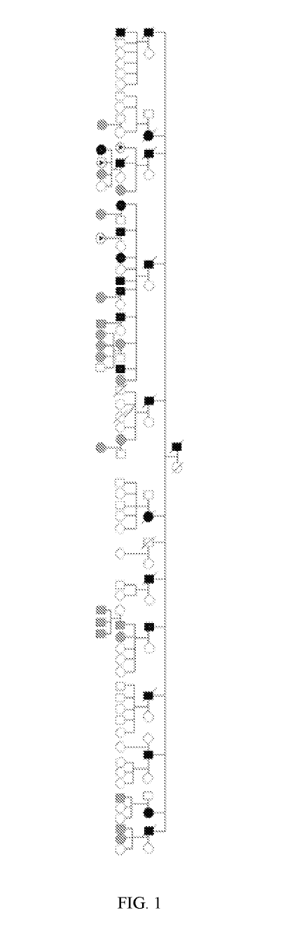

The Kindred CAD-2001 is white American, ascertained via a female index case, who presented with myocardial infarction (MI) at age 28. Coronary artery disease (CAD) risk factors included hypertension, diabetes mellitus, hypertriglyceridemia, and obesity. Evaluation revealed critical stenosis of two major coronary arteries, which led to coronary artery angioplasty and stenting. The kindred was subsequently extended (FIG. 1).

Among 46 blood relatives of the index case, 23 were diagnosed with early CAD (MI, angina, or sudden cardiac death) at or before age 50 (men) or 55 (women). Of these, 12 have died from CAD (mean age of death, 52 years). This familial clustering is noteworthy given that early CAD and early CAD death are uncommon in the general population.

All affected subjects trace their ancestry to a common ancestor. The ages of CAD onset ranged from 29 to 75, with a median age of 43. Among sibships in which all subjects are beyond the age threshold for onset of CAD, the offspring of single affected parents yielded 23 affected and 23 unaffected subjects, and male-to-female transmission is present. This extreme familial clustering and segregation of phenotypes within the kindred is unlikely to be explained by chance or multifactorial determination and provides strong evidence that early CAD is transmitted as a highly penetrant autosomal dominant trait. The familial clustering and pattern of inheritance of these clinical features were consistent with the effect of a highly penetrant autosomal dominant trait and suggested that the affected family members might share a common founder mutation.

Detailed clinical data were obtained for all available kindred members, including 11 living affected with early CAD, 12 free of early CAD at or beyond the age threshold of 50 years (men) and 55 (women), and 3 younger asymptomatic members (CAD phenotype unknown; mean age 30 years). Subjects were considered hypertensive if they were taking medication to treat hypertension, or if their blood pressure was greater than 140/90 mm Hg. Subjects were considered diabetic if they had a fasting blood sugar greater than 126 mg/dL. Informed consent was obtained from all subjects.

In family members with available clinical data, the clustering of the high cholesterol, diabetes and hypertension elements of metabolic syndrome with CAD was examined. Cardiac risk factors before or at presentation among affected subjects were surprisingly homogeneous, including marked hypertension in all, hypertriglyeridemia in all (mean 287.1 mg/dl, nl<150 mg/dl), low HDL in all (mean 35.1, nl>50 mg/dl) and type II diabetes in all (fasting blood glucose>126 mg/dl; or on drugs). All of the affected subjects met criteria of the NIH National Cholesterol Education Program for metabolic syndrome based on the presence of diabetes, high triglycerides, and hypertension. In contrast to these affected subjects, the twelve unaffected family members all had normal triglycerides (mean 100.5 mg/dl), close to normal HDL levels, and type 2 diabetes mellitus was absent. Finally, among younger subjects with CAD phenotype unknown, all 3 had high triglyceride levels, and were hypertensive.

All living family members were available for genetic studies, including 11 with early CAD. Exome analysis for the kindred in question and the Yale control exome database was produced at the W. M. Keck Facility of Yale University, as previously described. The Roche/Nimble-Gen 2.1M Human Exome Array covers 34.0 Mb of genomic sequence and about 180 000 exons of 18 673 protein-coding genes. Examined DNA was fragmented, ligated to linkers and fractionated by agarose gel electrophoresis. Extracted DNA was amplified by PCR and hybridized to the capture arrays. Resulting bound DNA was eluted, purified and amplified by ligation-mediated PCR. The PCR products were purified and sequenced on the Illumina DNA sequencing platform. Captured data was sequenced on the Illumina genome analyzer, followed by Image analysis and base calling. The resulting sequences were mapped to reference genome hg 19 using the Maqprogram SAMtools.

Sequence data was then processed using Maq software. SAMtools software was used to detect single nucleotide variants (SNVs). The SNVs were then filtered out against reference genome hg 19, as previously described. Filters were applied against a published database. A perl-based computer script was used to annotate variants based on protein effect, novelty, conservation and tissue expression.

The analysis showed a highly nonconservative novel single mutation in a previously characterized and functionally critical amino acid of Cela2a that was shared among all affected subjects and was absent in unaffected subjects. This amino acid is part of charge relay system plays a crucial role in this protein and its substitution has shown previously to cause loss of function. In addition, all subjects with type 2 diabetes, hypertension or hypertriglyceridemia, who were too young to develop CAD were carriers of this mutation. This mutation is absent in all public and Yale Exome databases.

### Example 2

### Independent Mutations in Cela2a Genes

Two thousand (2000) control exomes for mutations in Cela2a gene were screened. There was no single nonsynonymous mutation in this gene among controls. Then, the exome database of 30 selected index cases with early onset CAD (all before age 30) and metabolic syndrome were screened. These individuals have been excluded for mutations in known disease genes for CAD or metabolic traits. Two nonconservative mutations (1 splice site and one missense) were identified that were completely absent in the entire 10,000 exome database available at Yale.

### Example 3

### Tissue Expression of Cela2a

Cela2a is known as a pancreatic elastase. The findings herein prompted examination of its expression in different human tissues. Protein expression was examined in human and mouse tissues via western blot. Tissue lysates were prepared from human and mouse cadaveric tissues, and protein content was assessed in the lysates by ELISA Bradford assay. Lysate dilutions of equal protein concentration were loaded into a gel and fractionated via SDS-PAGE. Western blotting with an antibody was then used to determine protein expression in the tissue lysates. This showed variable expression of Cela2a in all tissues

### Example 3

### Plasma Activity of Cela2a

Plasma elastase activity levels in samples from the kindred were assessed via ELISA assay. Purified porcine protein was used to construct a standard curve. Plasma samples from all available subjects (all with a lab ID) were used in the assay. There was significantly reduced elastase activity in the plasma of mutation carriers vs. noncarriers.

### Example 4

### In Vivo and In Vitro Insulin Stimulation by Cela2a

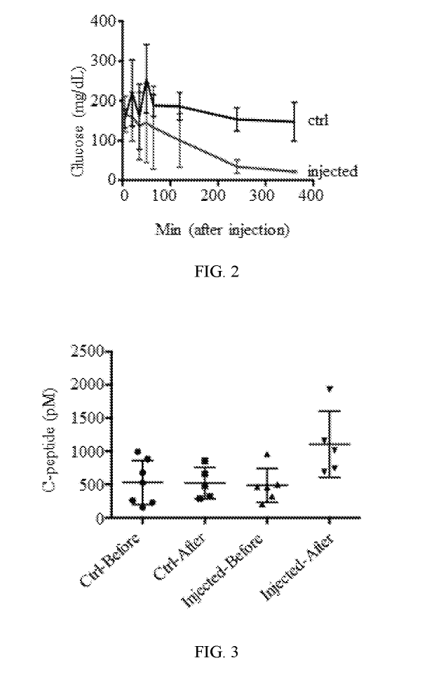

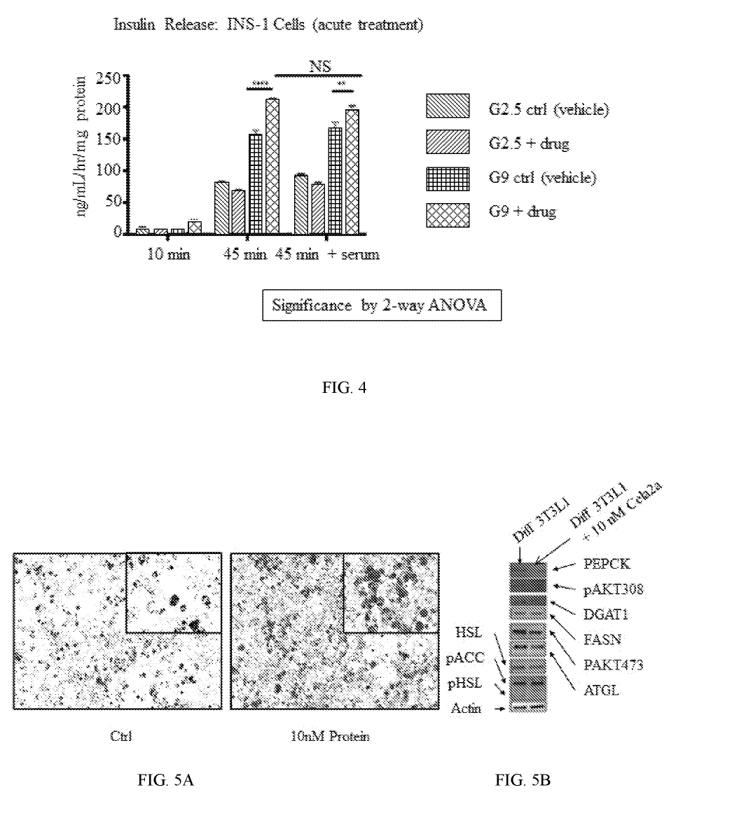

Mice on HID were injected with recombinant Cela2a protein and observed for 8 hrs. Plasma glucose dropped dramatically in mice injected with the protein compared to vehicle alone (FIG. 2). There was elevated C-peptide (FIG. 3) and inappropriately high insulin levels in mice after 8 hours. Subsequent INS-1 stimulation with the protein showed increased insulin secretion starting after 30 min (FIG. 4). Similar results were obtained when rat islet were stimulated with rhCela2a in presence of higher concentration of glucose. A plasma membrane protein that has the substrate binding site of the Cela2a (Ala-Ala-Pro-hydrophobic AA) was searched for and CD-36 was identified as well as platelet glycoproteins (see Example 7). CD-36 is an integral membrane protein found on the surface of diverse cells. Human islets express CD36 in the plasma membrane and insulin secretory granules. It plays a critical role for uptake of FA into beta-cells and insulin secretion. Its increased expression is associated with reduced insulin secretion. It has an extracellular Ala-Ala-Pro motif that can be cleaved by Cela2a and thus, the effect of Cela2a is likely mediated by disruption of CD36.

### Example 5

### Increased Adipogenesis and Reduced Lipolysis by Cela2a

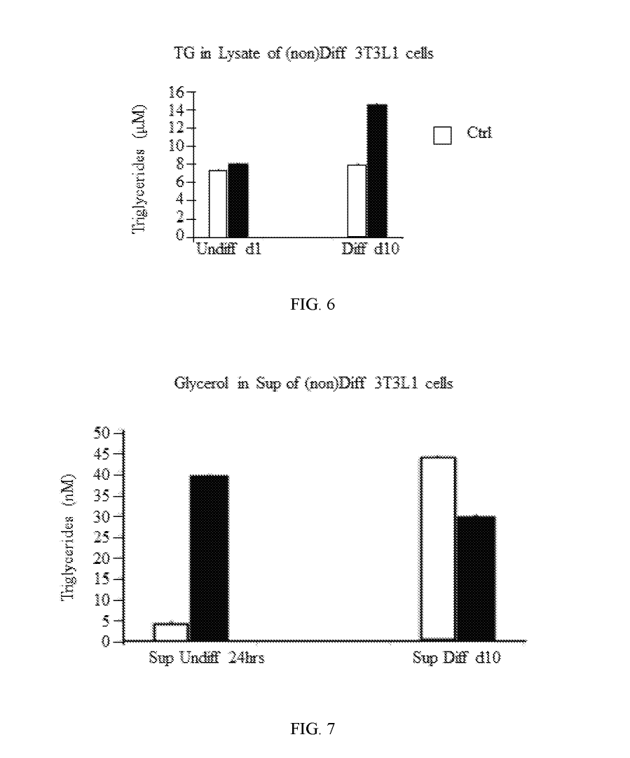

Cela2a inhibits lipolysis, an effect similar to insulin. The effect of Cela2a on adipogenic differentiation of 3T3L1 cells was examined. There was increased adipogenic differentiation in 3T3L1 cells treated with Cela2a vs. vehicle alone (FIG. 5A). Western blot analysis showed reduced expression of adipose triglyceride lipase (ATGL) and hormone-sensitive lipase (HSL) (FIG. 5B). This is associated with increased TAG deposition (FIG. 6) and reduced lipolysis, assayed by glycerol in supernatant (FIG. 7). Western blot analysis showed reduced expression of ATGL and HSL. Cela2a cleaves alphal-antitrypsin. A function that is defective in mutant Cela2a.

### Example 6

### Dissecting the Signaling Cascade Induced in Vitro by Cela2a Resulting in Insulin Stimulation

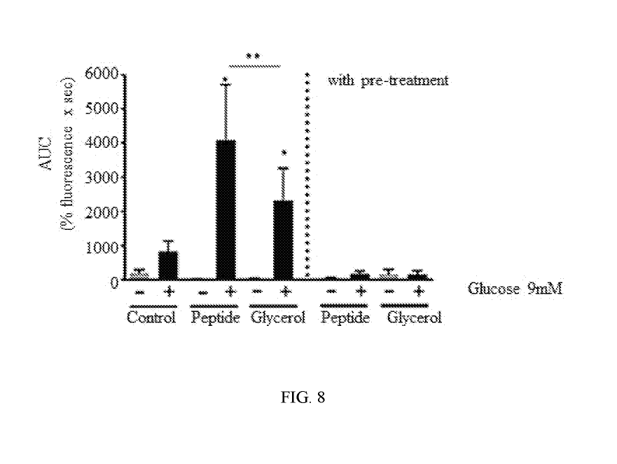

The calcium oscillation in Ins-1 cells treated with recombinant Cela2a was analyzed. Cells treated with Cela2a exhibited higher calcium oscillation peak compared with untreated cells and those treated only with the vehicle (FIG. 8).

### Example 7

### Cela2a Protects Against ADP-Induced Platelet Aggregation

Cela2a recognizes Ala-Ala-Pro-X(nonpolar AA) motif for its binding of the substrate and the catalytic activity. One such target in the gpIIb aka Integrin alpha-IIb(ITGA2B). Ala-Ala-Pro motif of the ITGA2B lies in its extracellular domain. Thus, Cela2a can cleave and inactivate this platelet receptor. Platelet aggregation was examined using human isolated platelets and WT and mutant Cela2a proteins. Treatment of platelets with wildtype Cela2a and more potently mutant Cela2a increased collagen induced platelet aggregation. Cela2a binds its antagonist, which includes alphal antitrypsin (AAT). We first examined interaction between AAT and Cela2a wildtype and mutant. Wildtype Cela2a binds AAT, while mutant Cela2a is incapable of binding it. Simultaneous treatment of platelets with wildtype and mutant Cela2a and AAT revealed that that AAT abolishes thrombotic effect of wildtype but not mutant Cela2a.

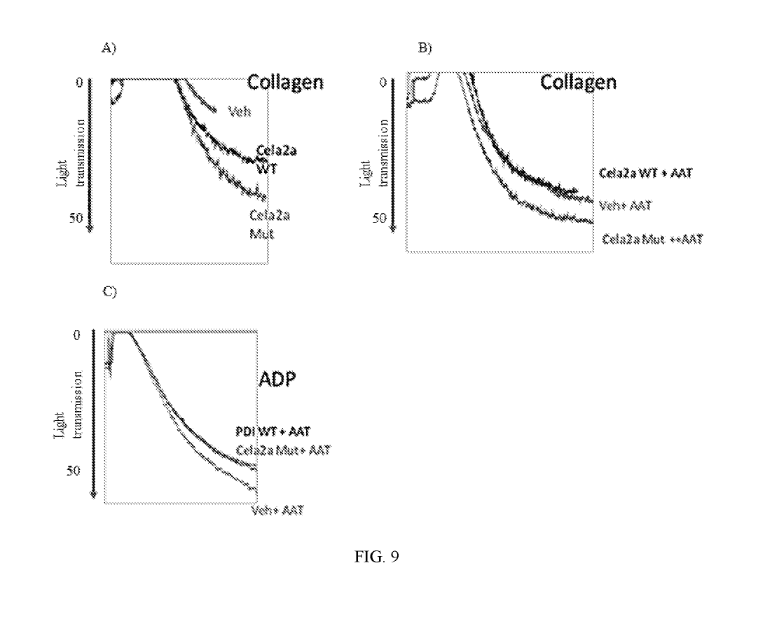

ADP induced platelet aggregation was then examined after simultaneous treatment of platelets with wildtype and mutant Cela2a and AAT. Both WT and mutant Cela2a were capable of preventing ADP-induced platelet aggregation compared to vehicle alone (FIGS. 9A-9C)

It is known that binding of ADP-activated platelets to HAEC is mediated by platelet GP IIb/IIIa. These findings suggest that cleavage of GP lib inhibits ADP activation of GP IIb/IIIa.

In conclusion Cela2a can be used in hypercoagulable states such as acute coronary artery syndrome together with ATT to prevent platelet aggregation

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A therapeutic composition comprising a Cela2a polypeptide in a pharmaceutically acceptable carrier.

2. The therapeutic composition of claim 1, wherein the composition is formulated for intravenous (IV) administration.

3. An expression vector comprising a Cela2a polynucleotide.

4. The expression vector of claim 3, wherein the Cela2a polynucleotide is operably linked to a constitutive promoter or an inducible promoter.

5. A host cell comprising the expression vector of claim 3.

6. A therapeutic composition comprising a Cela2a polypeptide or polynucleotide in an intracellular delivery vehicle.

7. A method of altering glucose or lipid metabolism in a subject, the method comprising administering to the subject an effective amount of a composition comprising a Cela2a polypeptide or polynucleotide, thereby altering glucose or lipid metabolism in the subject.

8. A method of treating atherosclerosis, diabetes, hypertension, or hypertriglyceridemia in a subject, the method comprising administering to the subject an effective amount of a composition comprising a Cela2a polypeptide or polynucleotide, thereby treating atherosclerosis, diabetes, hypertension, or hypertriglyceridemia in the subject.

9. The method of claim 7, wherein altering glucose or lipid metabolism comprises decreasing blood glucose level, stimulating insulin production, increasing adipogenesis, or reducing lipolysis.

10. A kit comprising a capture reagent that binds a Cela2a polypeptide or polynucleotide.

11. The kit of claim 10, further comprising the therapeutic composition of claim 1.

12. A method of detecting atherosclerosis and/or diabetes in a subject, the method comprising
measuring a level or a sequence of a Cela2a polypeptide or polynucleotide in a biological sample from the subject relative to a reference sequence,
wherein a decreased level of the Cela2a polypeptide or polynucleotide or presence of a mutation in the Cela2a polypeptide or polynucleotide sequence indicates presence of atherosclerosis and/or diabetes in the subject.

13. A method of identifying a subject at risk of developing atherosclerosis and/or diabetes, the method comprising
measuring a level or a sequence of a Cela2a polypeptide or polynucleotide in a biological sample from the subject relative to a reference level or reference sequence,
wherein a decreased level of the Cela2a polypeptide or polynucleotide or presence of a mutation in the Cela2a polypeptide or polynucleotide sequence indicates the subject is at risk of developing atherosclerosis and/or diabetes.

14. A method of treating atherosclerosis and/or diabetes in a subject, the method comprising
administering to the subject an effective amount of a Cela2a polypeptide, wherein the subject is pre-selected as having a mutation in a Cela2a polypeptide or polynucleotide relative to a reference sequence or a decreased level of a Cela2a polypeptide relative to a reference level,
thereby treating atherosclerosis and/or diabetes in the subject.

15. The method of claim 12, wherein the sequence or mutation in the Cela2a polypeptide or polynucleotide is detected using a kit comprising a capture reagent that binds a Cela2a polypeptide or polynucleotide.

16. The method of claim 12, wherein the step of measuring a sequence comprises measuring a sequence of a Cela2a polynucleotide.

17. The method of claim 12, wherein the mutation is non-conservative.

18. The method of claim 12, wherein the mutation is a loss-of-function mutation.

19. The method of claim 12, wherein the biological sample is blood or plasma.

20. The method of claim 7, wherein the composition is formulated in an intracellular delivery vehicle.

21. The method of claim 7, wherein the composition is administered to the subject by intravenous (IV) administration.

22. The method of claim 8, wherein the atherosclerosis is coronary artery disease (CAD) and/or the diabetes is type II diabetes.

23. The method of claim 7, wherein the subject is human.

24. A method of producing a Cela2a polypeptide, the method comprising
(a) expressing a recombinant Cela2a polypeptide in a host cell, and
(b) isolating the recombinant Cela2a polypeptide.

25. The method of claim 24, wherein the host cell comprises an expression vector comprising a Cela2a polynucleotide operably linked to a constitutive promoter or an inducible promoter.

26. The method of claim 13, wherein the sequence or mutation in the Cela2a polypeptide or polynucleotide is detected using a kit comprising a capture reagent that binds a Cela2a polypeptide or polynucleotide.

27. The method of claim 13, wherein the step of measuring a sequence comprises measuring a sequence of a Cela2a polynucleotide.

28. The method of claim 13, wherein the mutation is non-conservative.

29. The method of claim 13, wherein the mutation is a loss-of-function mutation.

30. The method of claim 13, wherein the biological sample is blood or plasma.

31. The method of claim 14, wherein the sequence or mutation in the Cela2a polypeptide or polynucleotide is detected using a kit comprising a capture reagent that binds a Cela2a polypeptide or polynucleotide.

32. The method of claim 14, wherein the step of measuring a sequence comprises measuring a sequence of a Cela2a polynucleotide.

33. The method of claim 14, wherein the mutation is non-conservative.

34. The method of claim 14, wherein the mutation is a loss-of-function mutation.

35. The method of claim 14, wherein the biological sample is blood or plasma.


SEQUENCE LISTING (2 sequences)

SEQ ID NO: 1 - protein, 269 aa, Homo sapien
MIRTLLLSTLVAGALSCGDPTYPPYVTRVVGGEEARPNSWPWQVSLQYSSNGKWYHTCGGSLIANSWVLTAAHCISSSRTYRVGLGRHNLYVAESGSLAVSVSKIVVHKDWNSNQISKGNDIALLKLANPVSLTDKIQLACLPPAGTILPNNYPCYVTGWGRLQTNGAVPDVLQQGRLLVVDYATCSSSAWWGSSVKTSMICAGGDGVISSCNGDSGGPLNCQASDGRWQVHGIVSFGSRLGCNYYHKPSVFTRVSNYIDWINSVIANN

SEQ ID NO: 2 - DNA, 934 bp, Homo sapien
gcttacagaactcccacggacacaccatgataaggacgctgctgctgtccactttggtggctggagccctcagttgtggggaccccacttacccaccttatgtgactagggtggttggcggtgaagaagcgaggcccaacagctggccctggcaggtctccctgcagtacagctccaatggcaagtggtaccacacctgcggagggtccctgatagccaacagctgggtcctgacggctgcccactgcatcagctcctccaggacctaccgcgtggggctgggccggcacaacctctacgttgcggagtccggctcgctggcagtcagtgtctctaagattgtggtgcacaaggactggaactccaaccaaatctccaaagggaacgacattgccctgctcaaactggctaaccccgtctccctcaccgacaagatccagctggcctgcctccctcctgccggcaccattctacccaacaactacccctgctacgtcacgggctggggaaggctgcagaccaacggggctgttcctgatgtcctgcagcagggccggttgctggttgtggactatgccacctgctccagctctgcctggtggggcagcagcgtgaaaaccagtatgatctgtgctgggggtgatggcgtgatctccagctgcaacggagactctggcgggccactgaactgtcaggcgtctgacggccggtggcaggtgcacggcatcgtcagcttcgggtctcgcctcggctgcaactactaccacaagccctccgtcttcacgcgggtctccaattacatcgactggatcaattcggtgattgcaaataactaaccaaaagaagtccctgggactgtttcagacttggaaaggtcacagaaggaaaataatataataaagtgacaactatgcaaatcaaaaaaaaaaaaaaa